(19) United States
(12) Patent Application Publication
(10) Pub. No.: US 2004/0122389 A1
(43) Pub. Date: Jun. 24, 2004

(54) Use of hygroscopic treatments to enhance dryness in an absorbent article

(76) Inventors: Tamara Lee Mace, Doraville, GA (US); Lon Michael Edelman, Alpharetta, GA (US); Christian Michael Trusock, Cumming, GA (US); Ali Yahiaoui, Roswell, GA (US); Rebecca Willey Griffin, Woodstock, GA (US)

Correspondence Address: KIMBERLY-CLARK WORLDWIDE, INC., 401 NORTH LAKE STREET, NEENAH, WI, 54956

(21) Appl. No.: 10/328,280

(22) Filed: Dec. 23, 2002

(51) Int. Cl. A61F 13/15; A61F 13/20

(52) U.S. Cl. 604/367

(57) ABSTRACT

An absorbent article includes a fluid permeable topsheet, a fluid impermeable backsheet, and a hygroscopic treatment. The hygroscopic treatment is protected from liquid contact under conditions of use and acts to reduce the water vapor present in the absorbent article environment, thereby reducing the relative humidity in the article. As a result, the article exhibits substantially reduced levels of hydration of the wearer's skin when in use which renders the skin less susceptible to the viability of microorganisms. The absorbent article may also include a ventilation layer between the absorbent body and the backsheet and/or a surge management layer between the absorbent body and the topsheet.

### BACKGROUND OF THE INVENTION

Many known diaper configurations employ absorbent materials located between a liquid pervious topsheet and a vapor and liquid impermeable backsheet. Such backsheets are well suited to prevent the migration of liquid waste from the absorbent materials to the outer garments of a wearer. Unfortunately, the use of liquid and vapor impermeable backsheets can result in a high degree of humidity within the diaper when in use which may result in relatively high skin hydration levels. The occlusive, moist environment inside diapers incorporating such backsheets has been suggested to promote the viability of microorganisms, including Candida albicans, which can undesirably lead to the onset of diaper dermatitis (diaper rash).

Diaper dermatitis can afflict almost every infant at some time during the diaper wearing years. The most severe form of this condition is usually caused by secondary infection with the fungi Candida albicans. Although other factors influence the pathogenesis of this fungi, one critical factor is the relative humidity within the diaper which is directly related to the occlusion or semi-occlusion of the diaper area.

In order to reduce the humidity level within diapers, breathable polymer films have been employed as outer covers for absorbent garments, such as disposable diapers. The breathable films are typically constructed with micropores to provide desired levels of liquid impermeability and air permeability. Other disposable diaper designs have been arranged to provide breathable regions in the form of breathable panels or perforated regions in otherwise vapor-impermeable backsheets to help ventilate the garment.

Conventional absorbent articles, such as those described above, have not been completely satisfactory. For example, articles which employ perforated films or breathable panels can exhibit excessive leakage of liquids from the article and can excessively soil the wearer's outer garments in the regions of the perforations or panels. In addition, when the absorbent material of the article becomes loaded with liquid, the wet absorbent can block the escape of moisture from the wearer's skin. Such absorbent garment designs have not been able to maintain a high level of breathability when wet to sufficiently reduce the hydration of the wearer's skin. As a result, the wearer's skin has remained susceptible to rashes, abrasion and irritation.

### SUMMARY OF THE INVENTION

The present invention relates to an absorbent article for absorbing fluids and exudates, such as urine. More particularly, the present invention relates to absorbent garments, such as disposable diapers and adult incontinence garments, which are configured to absorb body exudates while also helping to reduce the relative humidity in the environment of the article and to provide reduced skin hydration.

The present invention relates to an absorbent article comprising a topsheet or liner, a backsheet or outercover, and a hygroscopic treatment, wherein the hygroscopic treatment acts to reduce the water vapor present in the absorbent article environment, thereby reducing the relative humidity in the article. The reduction in humidity within the absorbent article environment generally results in reduced levels of skin hydration and a reduced viability of microorganisms. In one embodiment, the present invention may further include one or more materials positioned between the topsheet and the backsheet. The hygroscopic treatment is desirably applied to the liner and/or at least one of the one or more materials positioned between the topsheet and the backsheet such that the topsheet and/or at least one of the one or more materials positioned between the topsheet and the backsheet are highly hygroscopic.

In another embodiment, the hygroscopic treatment may be selectively applied to all or a portion of the liner and/or at least one of the one or more materials so as to produce a highly hygroscopic material at least in the portions of the article to which the hygroscopic treatment is applied. Desirably, the relative humidity in the environment is sufficiently low to reduce the onset of dermatitis. The absorbent article may further define a HLB of about 10 to about 20, and more desirably at least about 12.

In at least one embodiment of the present invention, the hygroscopic material may be comprised hydrogels, humectants, desiccants, surfactants, or blends or combinations thereof. Desirably, one or more components of the hygroscopic material is selected from the group consisting of siloxane polyethers, alkyl polyglycosides, water soluble cellulose derivatives, and polyethylene oxide derivatives of fatty acid esters or sorbitan esters, and more desirably the hygroscopic material may comprise Ahcovel Base N-62, DC193 Surfactant, and Glucopon 220UP.

In one aspect, the present invention relates to an personal care product which comprises a topsheet, a fluid impervious backsheet, and at least one material positioned between the topsheet and the backsheet, wherein at least a portion of one or more of the at least one materials and/or the topsheet is comprised of hygroscopic material, so as to absorb vapor from the environment of the personal care product to reduce the relative humidity therein. The hygroscopic material is such that it is protected from direct contact with liquid under conditions of use. Desirably, the relative humidity in the environment of the product is reduced so as to deter the onset of dermatitis, and more desirably, Candida Albicans. The absorbent article may further define a HLB of at least about 10. The relatively humidity in the environment of the product is desirably less than 81%, more desirably less than 75% and most desirably less than 65%. The absorbent article defines a C. albicans viability which is less than about 85 percent of the C. albicans viability of a control calculated according to a C. albicans Viability Test as set forth herein. In a particular embodiment, the C. albicans viability is less than about 80 percent and desirably less than about 60 percent of the C. albicans viability of the control calculated according to the C. albicans Viability Test. The absorbent article may further define a Skin Hydration Value of less than about 15 g/m²/hr calculated according to the Skin Hydration Test set forth herein.

In another aspect, the present invention relates to a personal care product which comprises a topsheet, a fluid impervious outercover, and at least one other material positioned between the topsheet and the outcover, wherein at least a portion of one of or more of the at least one other materials and/or the topsheet are highly hygroscopic to water vapor and is protected at least in part from direct liquid contact under conditions of use. The absorbent personal care product may further comprise a moisture barrier positioned between the topsheet and the at least one other material which is closest to the topsheet. The moisture barrier may act to further reduce the Skin Hydration Value. The absorbent article defines a Skin Hydration Value of less than about 15 g/m²/hr calculated according to the Skin Hydration Test set forth herein. In a particular embodiment, the absorbent article may define a Skin Hydration Value of less than about 14 g/m²/hr, and more desirably less than about 10 g/m²/hr calculated according to the Skin Hydration Test. The absorbent article may further define a HLB of at least about 10.

The present invention advantageously provides improved absorbent articles which exhibit substantially reduced levels of hydration of the wearer's skin when in use compared to conventional absorbent articles. The reduced level of skin hydration promotes drier, more comfortable skin and renders the skin less susceptible to the viability of microorganisms. Thus, wearer's of absorbent articles made according to the present invention have reduced skin hydration which can lead to a reduction in the incidence of skin irritation and rash.

### BRIEF DESCRIPTION OF THE DRAWINGS

The invention will be more fully understood and further advantages will become apparent when reference is made to the following detailed description of the invention and the accompanying drawings, in which:

FIG. 1 representatively shows a partially cutaway, top plan view of an absorbent article according to one embodiment of the invention; and

FIG. 2 representatively shows a sectional view of the absorbent article of FIG. 1 taken along line 2-2.

### DEFINITIONS

As used herein the following terms have the specified meanings, unless the context demands a different meaning, or a different meaning is expressed; also, the singular generally includes the plural, and the plural generally includes the singular unless otherwise indicated.

As used herein, all percentages, ratios and proportions are by weight unless otherwise specified.

As used herein, the term “biconstituent fibers” refers to fibers which have been formed from at least two polymers extruded from the same extruder as a blend. The term “blend” is defined below. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner. Bicomponent and biconstituent fibers are also discussed in the textbook Polymer Blends and Composites by John A. Manson and Leslie H. Sperling, copyright 1976 by Plenum Press, a division of Plenum Publishing Corporation of New York, IBSN 0-306-30831-2, at pages 273 through 277.

As used herein the term “blend” means a mixture of two or more polymers while the term “alloy” means a sub-class of blends wherein the components are immiscible but have been compatibilized. “Miscibility” and “immiscibility” are defined as blends having negative and positive values, respectively, for the free energy of mixing. Further, “compatibilization” is defined as the process of modifying the interfacial properties of an immiscible polymer blend in order to make an alloy.

As used herein, the terms “comprises”, “comprising” and other derivatives from the root term “comprise” are intended to be inclusive or open-ended terms that specify the presence of any stated features, elements, integers, steps, or components, but do not preclude the presence or addition of one or more other features, elements, integers, steps, components, or groups thereof. Accordingly, the term “comprising” encompasses the more restrictive terms “consisting essentially of” and “consisting of.”

As used herein, the term “conjugate fibers” refers to fibers which have been formed from at least two polymers extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. Conjugate fibers are taught in U.S. Pat. No. 5,108,820 to Kaneko et al., U.S. Pat. No. 5,336,552 to Strack et al., and U.S. Pat. No. 5,382,400 to Pike et al. For two component fibers, the polymers may be present in ratios of 75/25, 50/50, 25/75 or any other desired ratios. The fibers may also have shapes such as those described in U.S. Pat. Nos. 5,277,976 to Hogle et al., and 5,069,970 and 5,057,368 to Largman et al., hereby incorporated by reference in their entirety, which describe fibers with unconventional shapes.

As used herein, the term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

As used herein, the term “disposable” includes being disposed of after a single use and not intended to be washed and reused.

As used herein, the term “fabric” refers to all of the woven, knitted and nonwoven fibrous webs.

As used herein, the term “feminine hygiene products” includes sanitary napkins or pads, tampons and panty-liners and the like.

The terms “front” and “back” are used throughout this description to designate relationships relative to the garment itself, rather than to suggest any position the garment assumes when it is positioned on a wearer.

As used herein, the term “hygroscopicity” and other derivatives from the root term “hygroscopic” are intended to refer to the propensity of a material or component to interact with (or trapping of) water or water vapor.

As used herein, the term “hydrophilicity” and other derivatives from the root term “hydrophilic” are intended to refer to the solubility or dispersibility of chemicals in water liquid. In general, as used herein, the term “hydrophilic” refers to a material having a contact angle of water in air of less than 90 degrees.

As used herein, the terms “inward” and “outward” refer to positions relative to the center of a garment, and particularly transversely and/or longitudinally closer to or away from the longitudinal and transverse center of the garment.

As used herein, the term “layer” when used in the singular can have the dual meaning of a single element or a plurality of elements.

As used herein, the term “liquid” means a non-particulate substance and/or material that flows and can assume the interior shape of a container into which it is poured or placed.

As used herein, the term “liquid communication” means that liquid is able to travel from one layer to another layer, or one location to another within a layer.

As used herein, the terms “longitudinal” and “transverse” have their customary meanings. The longitudinal axis lies in the plane of the article when laid flat and fully extended and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

As used herein the term “microfibers” means small diameter fibers having an average diameter not greater than about 75 microns, for example, having an average diameter of from about 0.5 microns to about 50 microns, or more particularly, microfibers may have an average diameter of from about 2 microns to about 40 microns. Another frequently used expression of fiber diameter is denier, which is defined as grams per 9000 meters of a fiber and may be calculated as fiber diameter in microns squared, multiplied by the density in grams/cc, multiplied by 0.00707. A lower denier indicates a finer fiber and a higher denier indicates a thicker or heavier fiber. For example, the diameter of a polypropylene fiber given as 15 microns may be converted to denier by squaring, multiplying the result by its density value, e.g.,.89 g/cc, and multiplying by 0.00707. Thus, a 15 micron polypropylene fiber has a denier of about 1.42 (15²×0.89×0.00707=1.415). Outside the United States the unit of measurement is more commonly the “tex”, which is defined as the grams per kilometer of fiber. Tex may be calculated as denier/9.

As used herein the terms “nonwoven” and “nonwoven fabric or web” mean a web having a structure of individual fibers, filaments or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “personal care product” or “personal care absorbent product” means diapers, training pants, swim wear, absorbent underpants, baby wipes, incontinence products and devices, sanitary wipes, wet wipes, feminine hygiene products, absorbent pads, mortuary pads, veterinary pads, wound dressings and bandages, and the like.

As used herein, the term “skin” refers to the outermost exposed layer of a mammal's dermis or epidermis, and may be a wound.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

As used herein, the term “target area” or “target zone” refers to the area or position on a personal care product where an insult is normally delivered by a wearer or a test device in the case of certain product tests.

As used herein, a substantially fluid or liquid impermeable material is constructed to provide a hydrohead of at least about 60 centimeters (cm), desirably at least about 80 cm, and more desirably at least about 100 cm. A suitable technique for determining the hydrohead value is the Hydrostatic Pressure Test which is described in further detail herein below.

As used herein, a substantially vapor permeable material is constructed to provide a water vapor transmission rate (WVTR) of at least about 100 g/m²/24 hr, desirably at least about 250 g/m²/24 hr, and more desirably at least about 500 g/m²/24 hr. A suitable technique for determining the WVTR value is the Water Vapor Transmission Rate Test which is described in further detail herein below.

These terms may be defined with additional language in the remaining portions of the specification.

### TEST METHODS

Hydrostatic Pressure Test

The Hydrostatic Pressure Test is a measure of the liquid barrier properties of a material. In general, the Hydrostatic Pressure Test determines the height of water (in centimeters) in a column which the material will support before a predetermined amount of water passes through. A material with a higher hydrohead value indicates it is a greater barrier to liquid penetration than a material having a lower hydrohead value. The Hydrostatic Pressure Test is performed according to Method 5514—Federal Test Methods Standard No. 191A.

Water Vapor Transmission Test

A suitable technique for determining the WVTR (water vapor transmission rate) value of a material is as follows. For the purposes of the present invention, 3-inch diameter (76 millimeter) circular samples are cut from the test material and from a control material, Celguard® 2500 (Hoechst Celanese Corporation). Two or three samples are prepared for each material. Test cups used for testing are cast aluminum, flanged, 2 inches deep and come with a mechanical seal and neoprene gasket. The cups are distributed by Thwing-Albert Instrument Company, Philadelphia, Pa., under the designation Vapometer cup #681. One hundred milliliters of distilled water are poured into each Vapometer cup, and each of the individual samples of the test materials and control material are placed across the open top area of an individual cup. Screw-on flanges are tightened to form a seal along the edges of the cups leaving the associated test material or control material exposed to the ambient atmosphere over a 62 millimeter diameter circular area (an open, exposed area of about 30 cm²). The cups are then weighed, placed on a tray, and set in a forced air oven set at 100° F. (38° C.). The oven is a constant temperature oven with external air circulating through it to prevent water vapor accumulation inside. A suitable forced air oven is, for example, a Blue M Power-O-Matic 60 oven distributed by Blue M Electric Co. of Blue Island, Ill. After 24 hours, the cups are removed from the oven and weighed. The preliminary, test WVTR value is calculated as follows:

$$Test WVTR = \frac{[ ( grams weight loss over 24 hours ) \times 7571 ]}{24} ( g \text{/} {m}^{2} \text{/} 24 hours )$$

The relative humidity within the oven is not specifically controlled. Under predetermined set conditions of 100° F. and ambient relative humidity, the WVTR for Celguard 2500 has been determined to be 5000 g/m²/24 hours. Accordingly, Celguard 2500 is run as a control sample with each test. Celguard 2500 is a 0.0025 cm thick film composed of a microporous polypropylene.

Skin Hydration Test

Skin hydration values are determined by measuring total evaporative water loss (EWL) and can be determined by employing the following test procedure.

The test is conducted on partially toilet trained infants who have no lotions or ointments on the skin and have not been bathed within 2 hours prior to the test. Each infant tests one diaper during each test session. The test diapers include a test code and a control code. The test diapers (test code and control code) are randomized.

Each test diaper is weighed before and after use to verify the volume of liquid added into the diaper. A felt tip pen is employed to mark an “X” at the target zone inside the diaper, with the “X” positioned 6.5 inches below the top front edge of the diaper and centered side-to-side. The EWL measurements are taken with an evaporimeter, such as an Evaporimeter EP1 instrument distributed by Servomed AB, Stockholm, Sweden. Each test measurement is taken over a period of two minutes with EWL values taken once per second (a total of 120 EWL values). The digital output from the Evaporimeter EP1 instrument gives the rate of evaporative water loss (EWL) in g/m²/hr. Skin hydration values (SHV) are in units of total amount of water loss per unit area measured during the two-minute sampling period and are calculated as follows.

$$SHV ( g \text{/} {m}^{2} \text{/} hour ) = \frac{{\sum }_{n = 1}^{120} {( EWL )}_{n}}{120}$$

A preliminary skin hydration value measurement is taken after a 15-minute “dryout” period when the infant wears only a long T-shirt or dress and is in the supine position. The measurement is taken on the infant's lower abdomen, in a region corresponding to the target zone of the diaper, using the evaporimeter for the purpose of establishing the initial skin hydration value of the infant's skin at the diaper target zone. If the preliminary SHV is less than 10 g/m²/hour, a diaper is then placed on the infant. If the preliminary SHV is greater than 10 g/m²/hour, the “dryout” period is extended until a reading below 10 g/m²/hour is obtained. Prior to securing the diaper on the infant, a tube is positioned to direct a flow of liquid to hit the premarked target zone. Once the diaper is secured, 210 milliliters of adjusted 0.9 weight percent aqueous saline is added in three insults of 70 milliliters each at a rate of 15 milliliters/second with a 45 second delay between insults.

The infant wears the diaper for 60 minutes after which the diaper is removed and a test measurement of skin hydration is taken on the lower abdomen corresponding to the target zone mark of the diaper. The measurement is taken over a 2-minute period. The used diaper is then weighed. Relative humidity and temperature measurements can be taken within the diaper prior to the skin hydration measurements being taken. The test procedure is then repeated the next day for each infant using the diaper type (test or control) which the infant has not yet worn. The control diaper provides a standardized basis for comparing the performance of the diaper configuration being tested and evaluated.

Data is discarded for any infants which have added to the loading of saline solution, e.g. if the infants insult the diaper. The value reported for the mean net SHV (grams/m² in one hour) is the arithmetic mean for all infants of the post-wear skin hydration value, taken at the lower abdomen (target zone mark), minus the skin hydration value measured at the lower abdomen prior to placing the diaper on the infant (after “dryout” period). A separate mean net SHV is determined for the test code diapers and the control code diapers.

The net skin hydration value is determined as follows:

Net SHVᵢ=Y−Z

Where:

Y=skin hydration value measured at target zone mark of an individual infant

Z=baseline skin hydration value measured on the lower abdomen after “dryout” period prior to placing diaper on the infant

SHV₁=skin hydration value for individual infant

Then,

$$Mean Net SHV = \frac{{\sum }_{i = 1}^{N} Net {SHV}_{i} }{N}$$

Where: N=number of infants in study

The percent reduction in skin hydration is determined as follows:

$$\% Reduction = \frac{{\sum }_{i = 1}^{N} [ ( ( C - D ) \text{/} C ) \times 100 ]}{N}$$

Where:

C=Net SHVᵢ for control diaper code

D=Net SHVᵢ for test diaper code

N=number of infants in study

C. albicans Viability Test

The C. albicans Viability Test is a measure of the effect of absorbent garments, such as disposable diapers, on the viability of pathogenic microorganisms and, in particular, Candida albicans. In general, the C. albicans Viability Test involves inoculating delineated sites of each volar forearm of test subjects with a known suspension of C. albicans cells, covering the sites with full thickness patch from the absorbent garment, and determining the viability after a 24 hour period.

A full thickness test sample patch having a length of about 5 centimeters and a width of about 5 centimeters is cut from the target zone of each product to be tested. The target zone is generally that portion of the product intended to receive urine discharge from the wearer and typically includes portions of the intermediate and front waist sections of the product somewhat forward of the lateral centerline of the product. In a typical diaper configuration, the full thickness test sample patch includes the topsheet, absorbent body, backsheet and any intervening layers. Approximately 15 milliliters of a 0.9 weight percent saline solution is added to the test sample patch and allowed to soak in for 2 minutes before the samples are placed on the forearms of the test subjects. A test site area of about 6.15 square centimeters is marked on each of the test subject's volar forearms. Approximately 0.01 milliliters of a 0.9 weight percent saline solution containing a known suspension of C. albicans cells is delivered to the test site with micropipettes and the suspension is then spread uniformly across the test site. After air drying, the test site is covered with the test sample patch which is secured in position using adhesive tape completely surrounding the sample.

After 24 hours, the test sample patches are removed and a quantitative culture is obtained from the test site using the detergent scrub method set forth in “A New Method For Quantitative Investigation of Cutaneous Bacteria”, P. Williamson and A. M. Klingman, Journal of Investigative Dermatology, 45:498-503, 1965, the disclosure of which is hereby incorporated by reference in its entirety. Briefly, a sterile glass cylinder encompassing an area of 6.15 square centimeters is centered over the test site and held firmly to the skin. One milliliter of 0.1 weight percent Triton-x-100 in 0.075M phosphate buffer having a pH of 7.9 is pipetted into the glass cylinder and the area scrubbed for one minute using a sterile Teflon rod. The fluid is aspirated with a sterile pipette and a second milliliter of 0.1 weight percent Triton-x-100 in 0.075M phosphate buffer having a pH of 7.9 is added to the glass cylinder. The scrub step is repeated and the two washes are pooled. Each pooled sample is diluted in ten-fold steps with of 0.05 weight percent Triton-x-100 in 0.0375M phosphate buffer having a pH of 7.9. A 0.01 milliliter aliquot of each dilution is inoculated onto Sabourands agar containing antibiotics. Duplicate cultures are prepared and incubated at room temperature for 48 hours.

After incubation, the number of colony forming units are counted using standard microbiological methods. The C. albicans viability under a patch of the test sample can then be compared to the C. albicans viability under a control patch from a conventional absorbent article having a hygroscopic treatment.

Mocon Water Vapor Transmission Rate Test

A suitable technique for determining the WVTR (water vapor transmission rate) value of a material is the test procedure standardized by INDA (Association of the Nonwoven Fabrics Industry), number IST-70.4-99, entitled “STANDARD TEST METHOD FOR WATER VAPOR TRANSMISSION RATE THROUGH NONWOVEN AND PLASTIC FILM USING A GUARD FILM AND VAPOR PRESSURE SENSOR” which is incorporated by reference herein. The INDA procedure provides for the determination of WVTR, the permeance of the film to water vapor and, for homogeneous materials, water vapor permeability coefficient.

The INDA test method is well known and will not be set forth in detail herein. However, the test procedure is summarized as follows. A dry chamber is separated from a wet chamber of known temperature and humidity by a permanent guard film and the sample material to be tested. The purpose of the guard film is to define a definite air gap and to quiet or still the air in the air gap while the air gap is characterized. The dry chamber, guard film, and the wet chamber make up a diffusion cell in which the test film is sealed. The sample holder is known as the Permatran-W model 100K manufactured by Mocon/Modern Controls, Inc, Minneapolis, Minn. A first test is made of the WVTR of the guard film and air gap between an evaporator assembly that generates 100 percent relative humidity. Water vapor diffuses through the air gap and the guard film and then mixes with a dry gas flow which is proportional to water vapor concentration. The electrical signal is routed to a computer for processing. The computer calculates the transmission rate of the air gap and guard film and stores the value for further use.

The transmission rate of the guard film and air gap is stored in the computer as CalC. The sample material is then sealed in the test cell. Again, water vapor diffuses through the air gap to the guard film and the test material and then mixes with a dry gas flow that sweeps the test material. Also, again, this mixture is carried to the vapor sensor. The computer then calculates the transmission rate of the combination of the air gap, the guard film, and the test material. This information is then used to calculate the transmission rate at which moisture is transmitted through the test material according to the equation:

TR⁻¹_{test material}=TR⁻¹_{test material, guardfilm, airgap}−TR⁻¹_{guardfilm, airgap}

Calculations:

WVTR: The calculation of the WVTR uses the formula:

WVTR=Fρₛₐₜ(T)RH/APₛₐₜ(T)( 1−RH))

where:

F=The flow of water vapor in cc/min.,

ρₛₐₜ(T)=The density of water in saturated air at temperature T,

RH=The relative humidity at specified locations in the cell,

A=The cross sectional area of the cell, and,

ρₛₐₜ(T)=The saturation vapor pressure of water vapor at temperature T.

### DETAILED DESCRIPTION OF THE INVENTION

The following detailed description will be made in the context of a disposable diaper article which is adapted to be worn by infants about the lower torso. It is readily apparent, however, that the absorbent article of the present invention would also be suitable for use as other types of absorbent articles, training pants, absorbent underpants, baby wipes, incontinence products and devices, sanitary wipes, wet wipes, feminine hygiene products, absorbent pads, mortuary products, veterinary products, wound dressings and bandages, hygiene products and the like.

The absorbent articles of present invention advantageously exhibit a substantially reduced level of hydration of the wearer's skin in use when compared to conventional absorbent articles. Thus, wearers of absorbent articles of the different aspects of the present invention have reduced skin hydration which renders the skin less susceptible to the viability of microorganisms which can lead to a reduction in the incidence of skin irritation and rash. It has been discovered that the ability of the absorbent articles of the present invention to exhibit a low level of hydration on the wearer's skin during use depends, at least in part, on the amount of fluid in contact with the skin of the wearer. Moreover, it has been further discovered that the achievement of such low levels of skin hydration further depends on the ability of the product to maintain a low relative humidity in the environment of the product or the microclimate created between the product and a wearer (hereafter “in the product” or “the environment of the product”). The relative humidity of the environment of the product is intended to include that which is not only in the product itself, but also that area between the user and the article or product.

The ability of an absorbent article to achieve reduced levels of relative humidity in the environment of the product, has, for the purposes of this disclosure, been quantified as the C. albicans Viability Test. The amount of vapor in the environment may also be evaluated using the Skin Hydration Test or TEWL Test.

To achieve the desired low levels of skin hydration, the absorbent articles of the different aspects of the present invention may be constructed to define a relative humidity in the product of less than those experienced by a wearer of a product with no humidity reduction or skin hydration reducing agents, and generally desirably less than about 81%, more desirably less than about 75%, and even more desirably less than about 65%. Absorbent articles which exhibit a relative humidity greater than those above may undesirably result in increased levels of skin hydration. Such increased levels of skin hydration can render the skin more susceptible to the viability of microorganisms which can undesirably lead to an increase in the incidence of skin irritation and rash. Although the reduction of the relative humidity in the environment of the product which does not achieve the levels of reduction indicated above may provide for the deterrence of the onset of some forms of dermatitis, it may not deter or significantly deter the onset of C. albicans.

The ability of the absorbent articles of the present invention to exhibit a greater reduction in the liquid or vapor present in the environment of the articles has led to reduced levels of skin hydration. As indicated above, the reduction in liquid or vapor present in the environment of the article can be further facilitated through the incorporation of a vapor breathable outercover and/or a moisture barrier positioned between the topsheet or liner and the intake layer or acquisition material. The ability of an absorbent article to achieve a low level of skin hydration has, for the purposes of this application, been quantified as the Skin Hydration Value. As used herein, the term “Skin Hydration Value” refers to the value determined according to the Skin Hydration Test set forth above. In general, the Skin Hydration Value may be determined by measuring the evaporative water loss on the skin of test subjects after wearing a wetted absorbent article for a set period of time. In particular embodiments, the absorbent articles of the different aspects of the present invention may be constructed to define a Skin Hydration Value of less than about 15 g/m²/hr, desirably less than about 13 g/m²/hr, even more desirably less than about 10 g/m²/hr for improved performance. For example, the absorbent articles of the present invention may define a Skin Hydration Value of from about 0.1 to about 15 g/m²/hr and desirably from about 0.1 to about 13 g/m²/hr. Absorbent articles which exhibit Skin Hydration Values greater than those above can render the skin more susceptible to the growth of microorganisms which can undesirably lead to an increase in the incidence of skin irritation and rash.

The absorbent articles of the present invention may further exhibit reduced viability rates of microorganisms which can lead to a reduction in skin irritation. While not wishing to be bound by theory, it is hypothesized that the reduced viability of microorganisms is a direct result of the reduction in liquid or vapor, and thus the relative humidity, in the environment of the articles of the present invention. The ability of an absorbent article to achieve a low rate of viability of microorganisms has, for the purposes of this application, been quantified as the C. albicans viability value since it is hypothesized that the presence of Candida albicans is directly related to the incidence of irritation and, in particular, rash. As used herein, the term “C. albicans viability” refers to the value determined according to the Candida albicans Viability Test set forth above. The Candida albicans Viability Test, in general, is a comparison of the C. albicans viability under a patch of the test absorbent article to the C. albicans viability under a control patch from a conventional absorbent article having a nonbreathable outer cover, i.e. an outer cover having a WVTR of less than 100 g/m² per 24 hours.

In particular embodiments, the absorbent articles of the different aspects of the present invention may be constructed to define a C. albicans viability of less than about 85 percent, generally less than about 80 percent, desirably less than about 65 percent, more desirably less than about 40 percent, and even more desirably less than about 20 percent of the C. albicans viability of the control for improved performance. For example, the absorbent articles of the present invention may define a C. albicans viability of less than about 2.5, desirably less than about 2.0, and more desirably less than about 1.75 log of C. albicans colony forming units when inoculated with a suspension of about 5-7 log of C. albicans colony forming units according to the Candida albicans Viability Test.

Absorbent articles which exhibit C. albicans viability values greater than those above can undesirably lead to an increase in the incidence of skin irritation and rash. Desirably, the above C. albicans viability values are obtained without the incorporation of antimicrobial agents into the absorbent articles which can be perceived by consumers in a negative manner.

It has been discovered that acceptable, improved performance of absorbent articles can be achieved by selecting constructions having a combination of one or more of the above-described properties. For example, a given level of acceptable, improved performance may be achieved by employing an absorbent article which exhibits a Skin Hydration Value of less than about 15 g/m²/hr and a reduction of relative humidity in the environment of the article to a level of less than about 81%, and desirably a Skin Hydration Value of less than about 13 g/m²/hr and a reduction of relative humidity in the environment of the article to a level of less than about 65%.

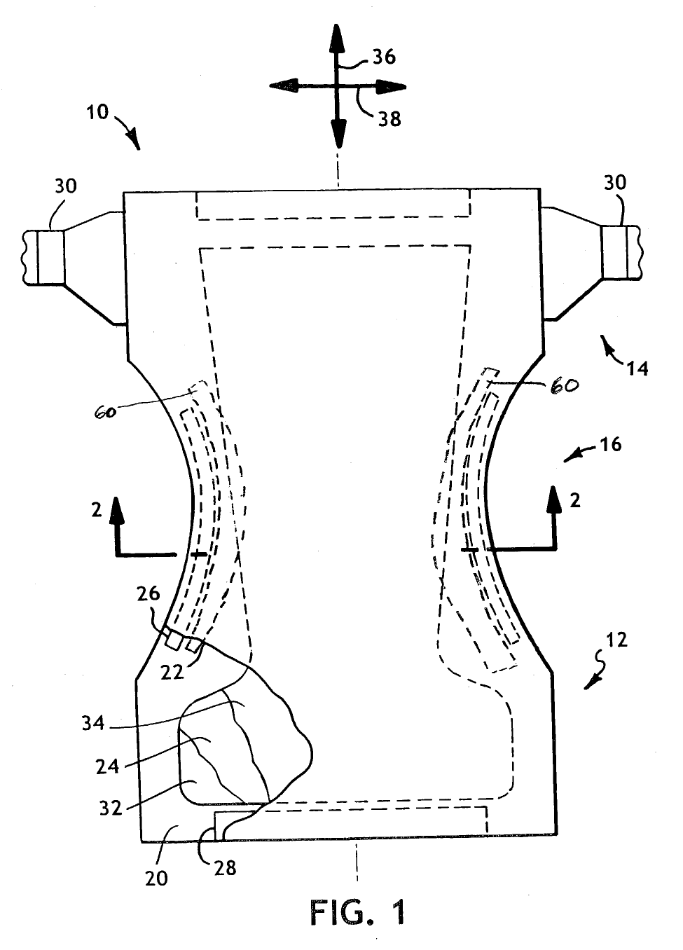

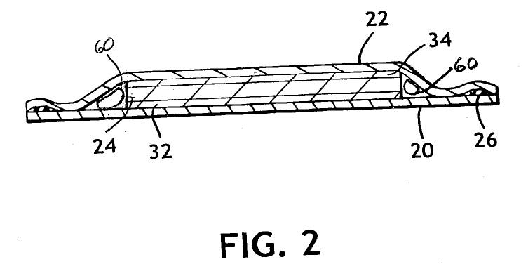

Examples of suitable constructions of absorbent articles for use in the present invention are described below and representatively illustrated in FIGS. 1 and 2. FIG. 1 is a representative plan view of an integral absorbent garment article, such as disposable diaper 10, of the present invention in its flat-out, uncontracted state (i.e., with all elastic induced gathering and contraction removed). Portions of the structure are partially cut away to more clearly show the interior construction of diaper 10, and the surface of the diaper which contacts the wearer is facing the viewer. FIG. 2 representatively shows a sectional view of the absorbent article of FIG. 1 taken along line 2-2. With reference to FIGS. 1 and 2, the disposable diaper 10 generally defines a front waist section 12, a rear waist section 14, and an intermediate section 16 which interconnects the front and rear waist sections. The front and rear waist sections include the general portions of the article which are constructed to extend substantially over the wearer's front and rear abdominal regions, respectively, during use. The intermediate section of the article includes the general portion of the article which is constructed to extend through the wearer's crotch region between the legs.

The absorbent article may include a vapor permeable backsheet 20, a liquid permeable topsheet 22 positioned in facing relation with the backsheet 20, and an absorbent body 24, such as an absorbent pad, which is located between the backsheet 20 and the topsheet 22. The backsheet 20 defines a length and a width which, in the illustrated embodiment, coincide with the length and width of the diaper 10. The absorbent body 24 generally defines a length and width which are less than the length and width of the backsheet 20, respectively. Thus, marginal portions of the diaper 10, such as marginal sections of the backsheet 20, may extend past the terminal edges of the absorbent body 24. In the illustrated embodiments, for example, the backsheet 20 extends outwardly beyond the terminal marginal edges of the absorbent body 24 to form side margins and end margins of the diaper 10. The topsheet 22 is generally coextensive with the backsheet 20 but may optionally cover an area which is larger or smaller than the area of the backsheet 20, as desired. The backsheet 20 and topsheet 22 are intended to face the garment and body of the wearer, respectively, while in use.

The permeability of the backsheet is configured to enhance the breathability of the absorbent article to reduce the hydration of the wearer's skin during use without allowing excessive condensation of vapor, such as urine, on the garment facing surface of the backsheet 20 which can undesirably dampen the wearer's clothes.

To provide improved fit and to help reduce leakage of body exudates from the diaper 10, the diaper side margins and end margins may be elasticized with suitable elastic members, such as single or multiple strands of elastic. The elastic strands may be composed of natural or synthetic rubber and may optionally be heat shrinkable or heat elasticizable. For example, as representatively illustrated in FIGS. 1 and 2, the diaper 10 may include leg elastics 26 which are constructed to operably gather and shirr the side margins of the diaper 10 to provide elasticized leg bands which can closely fit around the legs of the wearer to reduce leakage and provide improved comfort and appearance. Similarly, waist elastics 28 can be employed to elasticize the end margins of the diaper 10 to provide elasticized waists. The waist elastics are configured to operably gather and shirr the waist sections to provide a resilient, comfortably close fit around the waist of the wearer. In the illustrated embodiments, the elastic members are illustrated in their uncontracted, stretched condition for the purpose of clarity.

Fastening means, such as hook and loop fasteners 30, are employed to secure the diaper on a wearer. Alternatively, other fastening means, such as buttons, pins, snaps, adhesive tape fasteners, cohesives, mushroom-and-loop fasteners, or the like, may be employed.

The diaper 10 may further include other layers between the absorbent body 24 and the topsheet 22 or backsheet 20. For example, as representatively illustrated in FIGS. 1 and 2, the diaper 10 may include a ventilation or spacer layer 32 located between the absorbent body 24 and the backsheet 20 to insulate the backsheet 20 from the absorbent body 24 to improve air circulation and effectively reduce the dampness of the garment facing surface of the backsheet 20. The ventilation layer 32 may also assist in distributing fluid exudates to portions of the absorbent body 24 which do not directly receive the insult. The diaper 10 may also include a surge management layer 34 located between the topsheet 22 and the absorbent body 24 to prevent pooling of the fluid exudates and further improve air exchange and distribution of the fluid exudates within the diaper 10.

The diaper 10 may be of various suitable shapes. For example, the diaper may have an overall rectangular shape, T-shape or an approximately hour-glass shape. In the shown embodiment, the diaper 10 has a generally I-shape. The diaper 10 further defines a longitudinal direction 36 and a lateral direction 38. Other suitable diaper components which may be incorporated within absorbent articles of the present invention include containment flaps, waist flaps, elastomeric side panels, and the like which are generally known to those skilled in the art.

Examples of diaper configurations suitable for use in connection with the instant application which may include other diaper components suitable for use on diapers are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,176,672 issued Jan. 5, 1993, to Bruemmer et al.; U.S. Pat. No. 5,192,606 issued Mar. 9, 1993, to Proxmire et al., and U.S. Pat. No. 5,509,915 issued Apr. 23, 1996 to Hanson et al., the disclosures of which are herein incorporated by reference in their entirety.

The various components of the diaper 10 may be integrally assembled together employing various types of suitable attachment means, such as adhesive, sonic bonds, thermal bonds or combinations thereof. In the shown embodiment, for example, the topsheet 22 and backsheet 20 are assembled to each other and to the absorbent body 24 with lines of adhesive, such as a hot melt, pressure-sensitive adhesive. Similarly, other diaper components, such as the elastic members 26 and 28, fastening members 30, and ventilation and surge layers 32 and 34 may be assembled into the diaper article by employing the above-identified attachment mechanisms.

The backsheet 20 of the diaper 10, as representatively illustrated in FIGS. 1 and 2, is typically composed of a substantially vapor permeable material. The backsheet 20 may be generally constructed to be permeable to at least water vapor and may have a water vapor transmission rate of at least about 1000 g/m²/24 hr., desirably at least about 1500 g/m²/24 hr, more desirably at least about 2000 g/m²/24 hr., and even more desirably at least about 3000 g/m²/24 hr. For example, the backsheet 20 may define a water vapor transmission rate of from about 1000 to about 6000 g/m²/24 hr. Materials which have a water vapor transmission rate less than those above usually do not allow a sufficient amount of air exchange and can undesirably result in increased levels of skin hydration if no other means of humidity reduction within the diaper is available.

In one or more embodiments the backsheet 20 is also desirably substantially liquid impermeable. For example, the backsheet may be constructed to provide a hydrohead value of at least about 60 cm, desirably at least about 80 cm, and more desirably at least about 100 cm when subjected to the Hydrostatic Pressure Test. Materials which have hydrohead values less than those above can undesirably result in the strike through of liquids, such as urine, during use. Such fluid strike through can undesirably result in a damp, clammy feeling on the backsheet 20 during use.

The backsheet 20 may be composed of any suitable materials which either directly provide the above desired levels of liquid impermeability and air permeability or, in the alternative, materials which can be modified or treated in some manner to provide such levels. In one embodiment, the backsheet 20 may be a nonwoven fibrous web constructed to provide the required level of liquid impermeability. For example, a nonwoven web composed of spunbonded or meltblown polymer fibers may be selectively treated with a water repellent coating or laminated with a liquid impermeable, vapor permeable polymer film to provide the backsheet 20. In a particular embodiment of the invention, the backsheet 20 may comprise a nonwoven web composed of a plurality of randomly deposited hydrophobic thermoplastic meltblown fibers which are sufficiently bonded or otherwise connected to one another to provide a substantially vapor permeable and substantially liquid impermeable web. The backsheet 20 may also comprise a vapor permeable nonwoven layer which has been partially coated or otherwise configured to provide liquid impermeability in selected areas.

Examples of suitable materials for the backsheet 20 are also described in U.S. Pat. No. 5,482,765 issued Jan. 9, 1996 in the name of Bradley et al. and entitled “Nonwoven Fabric Laminate With Enhanced Barrier Properties”; U.S. Pat. No. 5,879,341 issued Mar. 9, 1999 in the name of Odorzynski et al. and entitled “Absorbent Article Having A Breathability Gradient”; U.S. Pat. No. 5,843,056 issued Dec. 1, 1998, in the name of Good et al. and entitled “Absorbent Article Having A Composite Breathable Backsheet”; and U.S. Pat. No. 6,309,736 issued Oct. 30, 2001, in the name of McCormack et al. and entitled “Low Gauge Films And Film/Nonwoven Laminates”, the disclosures of which are herein incorporated by reference in their entirety.

In a particular embodiment, the backsheet 20 is provided by a highly breathable laminate and more particularly by a microporous film/nonwoven laminate material comprising a spunbond nonwoven material laminated to a microporous film. The spunbond nonwoven comprises filaments of about 1.8 denier extruded from polypropylene and defines a basis weight of from about 17 to about 25 g/m². The film comprises a cast coextruded film having calcium carbonate-filled linear low polyethylene microporous core and ethylene vinyl acetate and Catalloy™ polypropylene (Catalloy™ 357P), available from Basell (having offices in Wilmington, Del.), blended skin layer having a basis weight of about 58 g/m² prior to stretching. The film is preheated, stretched and annealed to form the micropores and then laminated to the spunbond nonwoven. The resulting microporous film/nonwoven laminate based material has a basis weight of from about 30 to about 60 g/m² and a water vapor transmission rate of from about 3000 to about 5000 g/m²/24 hr. Examples of such film/nonwoven laminate materials are described in more detail in U.S. Pat. No. 6,309,736 issued Oct. 30, 2001, in the name of McCormack et al. and entitled “Low Gauge Films And Film/Nonwoven Laminates,” the disclosure of which was incorporated by reference above.

The topsheet 22, as representatively illustrated in FIGS. 1 and 2, suitably presents a bodyfacing surface which is compliant, soft feeling, and nonirritating to the wearer's skin. Further, the topsheet 22 may be less hydrophilic than the absorbent body 24, to present a relatively dry surface to the wearer, and may be sufficiently porous to be liquid permeable, permitting liquid to readily penetrate through its thickness. A suitable topsheet 22 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The topsheet 22 is suitably employed to help isolate the wearer's skin from liquids held in the absorbent body 24.

Various woven and nonwoven fabrics can be used for the topsheet 22. For example, the topsheet may be composed of a meltblown or spunbonded web of polyolefin fibers. The topsheet may also be a bonded-carded web composed of natural and/or synthetic fibers. The topsheet may be composed of a substantially hydrophobic material, and the hydrophobic material may, optionally, be treated with a surfactant or otherwise processed to impart a desired level of wettability and hydrophilicity. In a particular embodiment of the present invention, the topsheet 22 comprises a nonwoven spunbond, polypropylene fabric composed of about 2.2-2.8 denier fibers formed into a web having a basis weight of about 17 g/m² and a density of about 0.11 gram per cubic centimeter. Such a topsheet 22 may be surface treated with an effective amount of a surfactant such as about 0.3 weight percent of a surfactant commercially available from Uniqema under the trade designation AHCOVEL BASE N-62 (commonly referred to as Ahcovel).

In one embodiment, no surfactant will be added to or incorporated into the composite material of the present invention, however, in an alternative embodiment, the liner or topsheet 22 of the diaper 10 may also be treated with a surfactant to promote wettability of the liner, thereby promoting the wicking of moisture away from the surface of the user's skin and improved skin health conditions.

As noted above, in the alternative embodiment incorporating a surfactant, the fabric of the topsheet 36 may be surface treated with about 0.3 weight percent of a surfactant mixture which contains a mixture of AHCOVEL Base N-62 and GLUCOPON 220UP surfactant (commonly referred to Glucopon 220UP) in a 3:1 ratio based on a total weight of the surfactant mixture. Other possible classes of surfactants include MASIL SF 19 and DC 193 Surfactant. The AHCOVEL Base N-62 is purchased from Uniqema (a division of ICI, and having offices in New Castle, Del.), and includes a blend of hydrogenated ethoxylated castor oil and sorbitan monooleate. The GLUCOPON 220UP is purchased from Cognis Corporation and includes an alkyl polyglycoside. MASIL SF 19 and DC 193 Surfactant are purchased from BASF (Gurnee, Ill.), and Dow Corning (Midland, Mich.), respectively. MASIL SF 19 and DC 193 Surfactant are examples of typical ethoxylated polyalkylsiloxanes. The surfactant may be applied by any conventional means, such as saturation, spraying, printing, roll transfer, slot coating, brush coating, internal melt addition or the like. The surfactant may be applied to the entire topsheet 22 or may be selectively applied to particular sections of the topsheet 22, such as the medial section along the longitudinal centerline of the diaper, to provide greater wettability of such sections.

The absorbent body 24 of the diaper 10, as representatively illustrated in FIGS. 1 and 2, may suitably comprise a matrix of hydrophilic fibers, such as a web of cellulosic fluff, mixed with particles of a high-absorbency material commonly known as superabsorbent material. In a particular embodiment, the absorbent body 24 comprises a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent body 24 may comprise a laminate of fibrous webs and superabsorbent material or other suitable means of maintaining a superabsorbent material in a localized area.

The absorbent body 24 may have any of a number of shapes. For example, the absorbent core may be rectangular, I-shaped, or T-shaped. It is generally desired that the absorbent body 24 be narrower in the intermediate section than in the front or rear waist sections of the diaper 10. The absorbent body 24 may be provided by a single layer or, in the alternative, may be provided by multiple layers, all of which need not extend the entire length and width of the absorbent body 24. In a particular aspect of the invention, the absorbent body 24 can be generally T-shaped with the laterally extending cross-bar of the “T” generally corresponding to the front waist section 12 of the absorbent article for improved performance, especially for male infants. In the illustrated embodiments, for example, the absorbent body 24 across the front waist section 12 of the article has a cross-directional width of about 18 centimeters, the narrowest portion of the intermediate section 16 has a width of about 7.5 centimeters and in the rear waist section 14 has a width of about 11.4 centimeters.

The size and the absorbent capacity of absorbent body 24 should be compatible with the size of the intended wearer and the liquid loading imparted by the intended use of the absorbent article. Further, the size and the absorbent capacity of the absorbent body 24 can be varied to accommodate wearers ranging from infants through adults. In addition, it has been found that with the present invention, the densities and/or basis weights of the absorbent body 24 can be varied. In a particular aspect of the invention, the absorbent body 24 has an absorbent capacity of at least about 300 grams of synthetic urine.

In embodiments wherein the absorbent body 24 includes the combination of hydrophilic fibers and high-absorbency particles, the hydrophilic fibers and high-absorbency particles can form an average basis weight for the absorbent body 24 which is within the range of about 400 to about 900 g/m². In certain aspects of the invention, the average composite basis weight of such an absorbent body 24 is within the range of about 500 to about 800 g/m², and desirably is within the range of about 550 to about 750 g/m² to provide the desired performance.

To provide the desired thinness dimension to the various configurations of the absorbent article of the invention, the absorbent body 24 can be configured with a bulk thickness which is not more than about 0.6 centimeters. Desirably, the bulk thickness is not more than about 0.53 centimeters, and more desirably is not more than about 0.5 centimeters to provide improved benefits. The bulk thickness is determined under a restraining pressure of 0.2 psi (1.38 kPa).

The high-absorbency or superabsorbent material can be selected from natural, synthetic, and modified natural polymers and materials. The high-absorbency materials can be inorganic materials, such as silica gels, or organic compounds, such as crosslinked polymers.

Examples of synthetic, polymeric, high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful in the present invention.

The high absorbency material may be in any of a wide variety of physical forms. As a general rule, it is desired that the high absorbency material be in the form of discrete particles. However, the high absorbency material may also be in the form of fibers, flakes, rods, spheres, needles, or the like. In general, the high absorbency material is present in the absorbent body in an amount of from about 5 to about 90 weight percent, desirably in an amount of at least about 30 weight percent, and even more desirably in an amount of at least about 50 weight percent based on a total weight of the absorbent body 24. For example, in a particular embodiment, the absorbent body 24 may comprise a laminate which includes at least in part, and desirably at least about 50 weight percent and more desirably at least about 70 weight percent of high-absorbency material overwrapped by a fibrous web or other suitable means of maintaining the high-absorbency material in a localized area.

An example of high-absorbency material suitable for use in the present invention is HYSORB® P7050 polymer available from BASF. Other suitable superabsorbents may include, but are not limited to, DRYTECH® 2035 available from Dow Chemical Co. located in Midland, Mich., or FAVOR SXM 880 polymer obtained from Stockhausen, a business having offices in Greensboro, N.C.

Optionally, a substantially hydrophilic tissue wrapsheet (not illustrated) may be employed to help maintain the integrity of the structure of the absorbent body 24. The tissue wrap sheet is typically placed about the absorbent body over at least the two major facing surfaces thereof and composed of an absorbent cellulosic material, such as creped wadding or a high wet-strength tissue. In one aspect of the invention, the tissue wrap can be configured to provide a wicking layer which helps to rapidly distribute liquid over the mass of absorbent fibers comprising the absorbent body.

The absorbent body 24 of the different aspects of the present invention may further include a plurality of zones of high air permeability (not shown) which allow air and vapors to readily pass through the absorbent body 24 and through the vapor permeable backsheet 20 out of the diaper 10 into ambient air. A more detailed description and discussion of exemplary unitary components may be found in U.S. Pat. No. 6,152,906 issued Nov. 28, 2000 to Faulks et al.; U.S. Pat. No. 6,238,379 issued May 29, 2001, to Keuhn et al.; and U.S. Pat. No. 6,287,286 issued on Sep. 11, 2001 to Akin et al., the disclosures of which are incorporated by reference in their entirety.

As in conventional absorbent articles, due to the thinness of absorbent body 24 and the presence of high absorbency material within the absorbent body 24 of the present invention, the liquid uptake rates of the absorbent body 24, by itself, may be too low, or may not be adequately sustained over multiple insults of liquid into the absorbent body 24. To improve the overall liquid uptake and air exchange, a desired embodiment of the present invention may further include the previously mentioned additional porous, liquid-permeable layer of surge management material 34, as representatively illustrated in FIGS. 1 and 2. The surge management layer 34 is typically less hydrophilic than the absorbent body 24, and has an operable level of density and basis weight to quickly collect and temporarily hold liquid surges, to transport the liquid from its initial entrance point and to substantially completely release the liquid to other parts of the absorbent body 24. This configuration can help prevent the liquid from pooling and collecting on the portion of the absorbent garment positioned against the wearer's skin, thereby reducing the feeling of wetness by the wearer. The structure of the surge management layer 34 also generally enhances the air exchange within the diaper 10.

Various woven and nonwoven fabrics can be used to construct the surge management layer 34. For example, the surge management layer 34 may be a layer composed of a meltblown or spunbonded web of synthetic fibers, such as polyolefin fibers. The surge management layer 34 may also, for example, be a bonded-carded-web or an airlaid web composed of natural and synthetic fibers. The bonded-carded-web may, for example, be a thermally bonded web which is bonded using low melt binder fibers, powder or adhesive. The webs can optionally include a mixture of different fibers. Although the layer of surge material may itself be made up of one or more layers of materials, for purposes of this disclosure the surge layer shall be referred to, for descriptive purposes only, as one layer. The surge management layer 34 may be composed of a substantially hydrophobic material, and the hydrophobic material may optionally be treated with a surfactant or otherwise processed to impart a desired level of wettability and hydrophilicity. In a particular embodiment, the surge management layer 34 includes a hydrophobic, nonwoven material having a basis weight of from about 30 to about 120 g/m².

For example, in a particular embodiment, the surge management layer 34 may comprise a bonded-carded-web, nonwoven fabric which includes bicomponent fibers and which defines an overall basis weight of about 83 g/m². The surge management layer 34 in such a configuration can be a homogeneous blend composed of about 60 weight percent polyethylene/polyester (PE/PET), sheath-core bicomponent fibers which have a fiber denier of about 3 d and about 40 weight percent single component polyester fibers which have a fiber denier of about 6 d and which have fiber lengths of from about 3.8 to about 5.1 centimeters.

In the illustrated embodiments, the surge management layer 34 is desirably arranged in a direct, contacting liquid communication fashion with the absorbent body 24. The surge management layer 34 may be operably connected to the topsheet 22 with a conventional pattern of adhesive, such as a swirl adhesive pattern. In addition, the surge management layer 34 may be operably connected to the absorbent body 24 with a conventional pattern of adhesive. The amount of adhesive add-on should be sufficient to provide the desired levels of bonding, but should be low enough to avoid excessively restricting the movement of liquid from the topsheet 22, through the surge management layer 34 and into the absorbent body 24.

The absorbent body 24 is desirably positioned in liquid communication with surge management layer 34 to receive liquids released from the surge management layer, and to hold and store the liquid. In the shown embodiments, the surge management layer 34 comprises a separate layer which is positioned, at least in part, over another, separate layer comprising the absorbent body 24, thereby forming a dual-layer arrangement. The surge management layer 34 serves to quickly collect and temporarily hold discharged liquids, to transport such liquids from the point of initial contact and spread the liquid to other parts of the surge management layer 34, and then to substantially completely release such liquids into the layer or layers comprising the absorbent body 24.

The surge management layer 34 can be of any desired shape. Suitable shapes include for example, circular, rectangular, triangular, trapezoidal, oblong, dog-boned, hourglass-shaped, or oval. In certain embodiments, for example, the surge management layer can be generally rectangular-shaped. In the illustrated embodiments, the surge management layer 34 is coextensive with the absorbent body 24. Alternatively, the surge management layer 34 may extend over only a part of the absorbent body 24. Where the surge management layer 34 extends only partially along the length of the absorbent body 24, the surge management layer 34 may be selectively positioned anywhere along the absorbent body 24. For example, the surge management layer 34 may function more efficiently when it is offset toward the front waist section 12 of the garment. The surge management layer 34 may also be approximately centered about the longitudinal center line of the absorbent body 24.

Additional materials suitable for the surge management layer 34 are set forth in U.S. Pat. No. 5,486,166 issued Jan. 23, 1996 in the name of Ellis et al. and entitled “Fibrous Nonwoven Web Surge Layer For Personal Care Absorbent Articles And The Like”; U.S. Pat. No. 5,490,846 issued Feb. 13,1996 in the name of Ellis et al. and entitled “Improved Surge Management Fibrous Nonwoven Web For Personal Care Absorbent Articles And The Like”; and U.S. Pat. No. 5,364,382 issued Nov. 15, 1994 in the name of Latimer et al. and entitled “Absorbent Structure Having Improved Fluid Surge Management And Product Incorporating Same”, the disclosures of which are hereby incorporated by reference in their entirety.

Although, when present, a surge layer, a ventilating layer and/or zones of high air permeability may provide for the reduction of liquid and/or vapor moisture experienced by the user's skin, in some instances it is desirable to further reduce the relative humidity or moisture to which the user's skin is exposed. For example, these instances may occur where numerous insults of the same product have occurred, where the product is used over an extended period of time (e.g. incontinence pads), or where a product does not have a surge layer and/or regions or zones of high air permeability. Consequently, the present invention contemplates the addition of a hygroscopic treatment to the absorbent article, such that the hygroscopic treatment reduces the water vapor present in the absorbent article environment, thereby reducing the relative humidity in the article. Although a surge layer is not required in the present invention, a desired embodiment of the present invention includes a surge layer positioned between a fluid permeable topsheet and a fluid impermeable backsheet. As indicated above, the present invention further includes the addition of a hygroscopic treatment. The treatment may, for example, be applied to the topsheet and/or one or more of the other materials or layers that may be desirably included so that part of the topsheet and/or all or a part of at least one of the other materials (depending on their function and location) are highly hygroscopic. It is contemplated that the hygroscopic treatment may be selectively applied to a portion of the topsheet and/or all or a portion of the other materials or layers that may be included in the article or product, so as to produce a highly hygroscopic material at least in the portions or regions of the topsheet and/or the materials to which the hygroscopic treatment is applied. Furthermore, it is recognized that the hygroscopic treatment may be applied at different levels or in different amounts to different portions of the absorbent article. For example, depending on the embodiment in question, it may be considered desirable to place more of the treatment on one of the materials or layers in the article than on the topsheet. For example, one or more of the materials or layers in the article other than the topsheet or outercover may in some embodiments be treated with from about 0.3% to about 30%, desirably from about 0.3% to about 15%, and even desirably from about 0.5% to about 3%, add-on of hygroscopic treatment, and the liner or topsheet may be treated with from about 0.3% to about 10%, and desirably from about 1% to about 5% add-on of hygroscopic treatment. Further still, different regions of the components of the can be treated with different add-on levels than other regions of the article and/or the component itself.

In addition to being able to be applied to the liner and/or one or more other materials of the article, the hygroscopic treatment may be applied to or otherwise included in an absorbent article in any suitable manner provided that the treatment is isolated or otherwise protected from a liquid environment, insult, or the like. That is, while the hygroscopic treatment is highly wettable, if exposed to a liquid, the treatment will become saturated and will not be able to function in that its moisture or vapor attraction be diminished or non-existent. Accordingly, while the hygroscopic treatment may be present within or an absorbent article anywhere which offers such protective conditions, in at least one embodiment, the hygroscopic treatment may be present in a vapor permeable container, such as pouch 60 in FIGS. 1 and 2. The pouches 60 should be liquid impermeable, but vapor permeable to meet the conditions described above. Desirably such a vapor permeable container will have a Mocon value (as determined in accordance with the Mocon Water Vapor Transmission Rate Test discussed above) of at least about 6,000, and more desirably at least about 10,000, and even more desirably at least about 30,000. Again, while a vapor permeable container, such as pouch 60, will protect the hygroscopic treatment from becoming saturated or inundated as a result of contact with a liquid, the vapor permeable container will still allow the hygroscopic treatment to absorb moisture in the form of vapor from the environment of the product. It will be appreciated that the extraction of moisture (e.g. vapor) from the environment of the product will reduce the relative humidity and Skin Hydration Value of the product. While the pouch 60 is shown as being below the wearer side of the topsheet or liner, it will be appreciated that the pouch may be anywhere within the article or on the topsheet provided that the treatment itself is at least in part protected from contact with a fluid under conditions of use. It will be appreciated that for ease of manufacturing, there may be instances where a hygroscopic treatment is applied to all portions of a material or component of an article even though some of the hygroscopic treatment thereon will be exposed to direct contact with a fluid upon insult. While this may occur as a result of manufacturing preferences, it should be appreciated that such a material or component should still be considered to be within the present invention provided at least some of the hygroscopic treatment is intended to be protected from direct contact with a liquid under conditions of use.

It will be further appreciated that while reference was made to a pouch above, any other suitable configuration including, for example, tubes, spheres, envelopes, or the like which have hygroscopic treatment therein may be used. Again, it will be appreciated, the more surface area of hygroscopic treatment which is exposed, the faster the relative humidity or Skin Hydration Value may be reduced.

As noted above, while the hygroscopic treatment of the present invention may be restricted in placement within or on an article or material because of the potential exposure to liquids, the hygroscopic treatment need not be in a vapor permeable container (e.g., a pouch or tube) specifically designed therefore, but rather may be anywhere in or on a product that offers the treatment the protection from liquids discussed above. That is, in one or more embodiments, the hygroscopic treatment may, for example, be applied to the back of a spacer layer, on the outside of a containment flap, on the inside of the outercover, or on the surface of fibers.

While the hygroscopic treatment may be present in or on the product in a variety of locations, it is desired that it be located as close to the skin of the wearer as possible. That is, while not wishing to be bound by theory, it is believed that the closer the hygroscopic treatment is to the skin the better the results (e.g. the more likely the relative humidity or Skin Hydration Value is to drop) as the hygroscopic treatment will tend to attract moisture (e.g. vapor) from the skin or environment between the skin and the product as opposed to from the absorbent core.

Any material which exhibits the above described characteristics may be a hygroscopic treatment. Suitable hygroscopic treatments include, but are not limited to, hydrogels, humectants, desiccants, surfactants, or blends or combinations thereof. Other specific examples of suitable hygroscopic treatments include, for example, clays, salts, and materials having a hygroscopic treatment applied thereto.

An embodiment of the present invention may include a hygroscopic material which has a Hydrophilic-Lipophilic Balance (HLB) of about 10 to about 20, and even more desirably a material having a HLB of at least about 12 to about 20. As above, the hygroscopic material may include, for example, include hydrogels, humectants, desiccants, surfactants, or blends or combinations thereof. In an exemplary embodiment, one or more components of a hygroscopic material may be selected from the group consisting of siloxane polyethers, alkyl polyglycosides, water soluble cellulose derivatives, and polyethylene oxide derivatives of fatty acid esters or sorbitan esters. A desired embodiment of the hygroscopic material includes AHCOVEL BASE N-62, DC193 Surfactant, and GLUCOPON 220UP.

Further, while the absorbent article should be designed such that the hygroscopic treatment is not in direct contact with a fluid, it will be appreciated that in some embodiments a fluid may eventually wick through the article or material in such a manner that some of the fluid contacts the hygroscopic treatment. While the contact is a possibility in those embodiments that do not use liquid impermeable pouches or the like, such contact is not desired and is desirably designed to occur only after the normal use period of the article after unusually large or multiple insults. Such prolonged use after such an insult is typically beyond the conditions in which the hygroscopic treatment is designed or in which the treatment can provide a significant benefit. That is, while it is desired that the hygroscopic treatment be protected so as to avoid any contact with a liquid, such as in a liquid impermeable pouch or the like, the protection need not be complete isolation of the treatment as, in some cases, depending on the length of time the article is used or worn, the hygroscopic material may eventually come in contact with or be exposed to liquid, but the absorbent article is desirably designed and configured such that this contact, if any, does not occur immediately upon insult, but rather after prolonged or extended use of the article.

Another embodiment of the present invention is directed to a personal care having a topsheet, a fluid impervious backsheet, and at least one material, the at least one material being positioned between the topsheet and the backsheet. At least a portion of the topsheet and/or at least a portion of one or more of the at least one materials are comprised at least in part of hygroscopic material. The hygroscopic material is desirably protected from direct contact with liquid so as to allow it to better absorb vapor from the environment of the personal care product thereby reducing the relative humidity therein.

Another embodiment of the present invention is directed to a personal care product capable of lowering the relative humidity of the product environment so as to deter the onset of dermatitis. The personal care product generally includes a topsheet, a fluid impervious outercover, and at least one other material positioned between the topsheet and the outercover. At least one portion of one or more of the at least one other materials and/or the topsheet should be highly hygroscopic to water vapor and is protected at least in part from direct liquid contact under conditions of use such that the personal care product defines a Skin Hydration Value of less than about 15 g/m²/hr calculated according to a Skin Hydration Test set forth herein.

The different embodiments of the present invention, as representatively illustrated in FIGS. 1 and 2, advantageously provide improved absorbent articles which exhibit substantially reduced levels of hydration of the wearer's skin when in use compared to conventional absorbent articles. The reduced levels of skin hydration promote drier, more comfortable skin and render the skin less susceptible to the viability of microorganisms. Thus, wearers of absorbent articles made according to the present invention may have reduced skin hydration which can lead to a reduction in the incidence of skin irritation and rash.

While various patents and other reference materials have been incorporated herein by reference, to the extent there is any inconsistency between incorporated material and that of the written specification, the written specification shall control. In addition, while the invention has been described in detail with respect to specific embodiments thereto, it will be readily apparent to those skilled in the art that various alterations, modifications and other changes can be made without departing from the spirit and scope of the present invention. It is therefore intended that the claims cover or encompass all such alterations, modifications and/or changes.

We claim:

1. An absorbent article comprising:
a fluid permeable topsheet;
a fluid impermeable backsheet; and
a hygroscopic treatment;
wherein the hygroscopic treatment is at least in part protected from liquid contact under conditions of use;
wherein the hygroscopic treatment reduces the water vapor present in the absorbent article environment, thereby reducing the relative humidity in the absorbent article environment.

2. The absorbent article of claim 1 further comprising one or more materials positioned between the topsheet and the backsheet.

3. The absorbent article of claim 2, wherein the hygroscopic treatment is applied to the topsheet and/or at least one of the one or more materials such that the topsheet and/or at least one of the one or more materials are highly hygroscopic at least in part.

4. The absorbent article of claim 2, wherein the hygroscopic treatment may be selectively applied to at least a portion of the topsheet and/or at least one of the one or more materials, so as to produce one or more highly hygroscopic materials at least in the portions of the article to which the hygroscopic treatment is applied.

5. The absorbent of claim 3, wherein the hygroscopic treatment may be applied at different levels to different portions of the absorbent article.

6. The absorbent article of claim 4, wherein the highly hygroscopic material has a HLB of about 10 to about 20.

7. The absorbent article of claim 4, wherein the highly hygroscopic material has a HLB of at least about 12.

8. The absorbent article of claim 1, wherein the hygroscopic treatment comprises hydrogels, humectants, desiccants, surfactants, or blends or combinations thereof.

9. The absorbent article of claim 1 further comprises a vapor permeable container; wherein the vapor permeable container is has hygroscopic treatment enclosed therein.

10. The absorbent article of claim 9, wherein the vapor permeable container is liquid impermeable.

11. The absorbent article of claim 9, wherein the vapor permeable container has a Mocon value of at least about 6,000.

12. The absorbent article of claim 9, wherein the vapor permeable container has a Mocon value of at least about 10,000.

13. The absorbent article of claim 1, wherein the hygroscopic treatment is selected from the group consisting of siloxane polyethers, alkyl polyglycosides, water soluble cellulose derivatives, and polyethylene oxide derivatives of fatty acid esters or sorbitan esters.

14. The absorbent article of claim 1, wherein the hygroscopic treatment comprises Ahcovel Base N-62, DC193 Surfactant, and Glucopon 220UP.

15. The absorbent article of claim 1, wherein the relative humidity in the environment of the article is sufficiently low to deter the onset of dermatitis.

16. The absorbent article of claim 11, wherein the hygroscopic treatment reduces the relative humidity within the environment of the article such that the growth of Candida Albicans is deterred.

17. The absorbent article of claim 2, wherein at least one of the one or more materials is treated with about 0.3% to about 30% add-on of hygroscopic treatment.

18. The absorbent article of claim 2, wherein at least one of the one or more materials is treated with about 0.3% to about 15% add-on of hygroscopic treatment.

19. The absorbent article of claim 2, wherein at least one of the one or more materials is treated with about 0.5% to about 3% add-on of hygroscopic treatment.

20. The absorbent article of claim 1, wherein at least a portion of the topsheet is treated with about 0.3% to about 10% add-on of hygroscopic treatment.

21. The absorbent article of claim 1, wherein at least a portion of the topsheet is treated with about 1% to about 5% add-on of hygroscopic treatment.

22. The absorbent article of claim 1, wherein the topsheet is selected from woven fabrics, knit fabrics, nonwoven fabrics, foams, film-like materials and paper materials.

23. The absorbent article of claim 1, wherein the article comprises a personal care product.

24. The absorbent article of claim 23, wherein the personal care product is selected from a diaper, training pant, absorbent underpant, adult incontinence product, sanitary wipe, wet wipe, feminine hygiene product, wound dressing, bandage, mortuary product, veterinary product, hygiene product and absorbent product.

25. A personal care product comprising:
a topsheet;
a fluid impervious backsheet; and
at least one material, the at least one material being positioned between the topsheet and the backsheet;
wherein at least a portion of at least one of the at least one materials and/or the topsheet are comprised at least in part of hygroscopic material, so as to absorb vapor from the environment of the personal care product to reduce the relative humidity therein; and wherein the hygroscopic material is protected from direct contact with liquid under conditions of use.

26. The personal care product of claim 25, wherein the hygroscopic material comprises hydrogels, humectants, desiccants, surfactants, or blends or combinations thereof.

27. The personal care product of claim 25, wherein the hygroscopic material is selected from the group consisting of siloxane polyethers, alkyl polyglycosides, water soluble cellulose derivatives, and polyethylene oxide derivatives of fatty acid esters or sorbitan esters.

28. The personal care product of claim 25, wherein the hygroscopic material comprises Ahcovel Base N-62, DC193 Surfactant, and Glucopon 220UP.

29. The personal care product of claim 25, wherein the relative humidity of the environment of the product is reduced such that the onset of dermatitis is deterred.

30. The personal care product of claim 25, wherein the relative humidity in the environment of the product is sufficiently low so as to deter the onset of Candida Albicans.

31. The personal care product of claim 25, wherein the relative humidity in the product is less than about 81%.

32. The personal care product of claim 25, wherein the relative humidity in the product is less than about 75%.

33. The personal care product of claim 25, wherein the relative humidity in the product is less than about 65%.

34. The personal care product of claim 25, wherein the at least one material is located in one or more regions of the product.

35. The personal care product of claim 34, wherein at least one of the regions of at least one of the at least one material is highly hygroscopic.

36. The personal care product of claim 25, wherein the personal care product is selected from a diaper, training pant, absorbent underpant, adult incontinence product, sanitary wipe, wet wipe, feminine hygiene product, wound dressing, bandage, and mortuary and veterinary wipe, hygiene product and absorbent product.

37. A personal care product capable of lowering the relative humidity of the product environment so as to deter the onset of dermatitis, said personal care product comprising:
a topsheet;
a fluid impervious outercover; and
at least one other material positioned between the topsheet and the outercover;
wherein at least one portion of one or more of the at least one other materials and/or said topsheet is highly hygroscopic to water vapor and is protected at least in part from direct liquid contact under conditions of use, and wherein said personal care product defines a Skin Hydration Value of less than about 15 g/m²/hr calculated according to a Skin Hydration Test set forth herein.

38. The personal care product of claim 37 further comprising a moisture barrier positioned between said topsheet and a first of said at least one other material, said moisture barrier further reducing the Skin Hydration Value as set forth herein.

39. The personal care product of claim 37, wherein said at least one portion of said topsheet and/or one or more of the at least one other materials has a HLB of at least about 10.

40. The personal care product of claim 37, wherein the personal care product is selected from a diaper, training pant, absorbent underpant, adult incontinence product, sanitary wipe, wet wipe, feminine hygiene product, wound dressing, bandage, and mortuary product, veterinary product, hygiene product and absorbent product.
